(19) United States
(12) Patent Application Publication
(10) Pub. No.: US 2023/0101125 A1
(43) Pub. Date: Mar. 30, 2023

(54) Systems And Method For Optogenetically Controlling Insulin Secretion For Treating Type 1 Diabetes

(71) Applicant: Board of Trustees of Michigan State University, East Lansing, MI (US)

(72) Inventors: Ping Wang, Okemos, MI (US); Wen LI, Okemos, MI (US); Aitor Aguirre, Okemos, MI (US)

(21) Appl. No.: 17/903,156

(22) Filed: Sep. 6, 2022

Related U.S. Application Data
(60) Provisional application No. 63/249,670, filed on Sep. 29, 2021.

(51) Int. Cl. A61N 5/06; A61K 35/39

(52) U.S. Cl. CPC A61N 5/0601; A61K 35/39; A61N 2005/0626; A61N 2005/0651; A61N 2005/0663; A61N 2005/0643

(57) ABSTRACT

A method and system for of treating type 1 includes implanting genetically modified islet cells under a capsule of or within an organ, implanting a microsystem adjacent the islet cells, said microsystem, comprising a light emitting diode stimulator comprising a plurality of light emitting diodes, determining a glucose level in a body and controlling the microsystem to selectively illuminate the islet cells to secrete insulin or glucagon or both based on the glucose level.

### CROSS-REFERENCE TO RELATED APPLICATION

This application claims the benefit of U.S. Provisional Application No. 62/249,670, filed on Sep. 29, 2021. The entire disclosure of the above application(s) is (are) incorporated herein by reference.

### FIELD

The present disclosure relates to treating Type 1 diabetes and, more specifically, to optogenenetically treating Type 1 diabetes by stimulating secretion of insulin from grafted stem cell derived β-cells (SC-β-cells).

### BACKGROUND

Type 1 diabetes (T1D) arises from the selective and progressive autoimmune destruction of p-cells within the islets of Langerhans in the pancreas. Type 1 diabetes affects 20-40 million people worldwide, and accounts for 5% of deaths annually. While insulin treatment can control hyperglycemia and delay the progression of some complications, it does not cure T1D. Pancreatic islet cell transplantation (Tx) has lifesaving potential for curing T1D patients. However, even with the success of the advanced immunosuppressive protocol, only 20% of patients remain insulin independent 3 years after islet cell transplantation. Significant islet destruction after the Tx procedure contributes to such poor outcomes. Another significant problem hampering the successful clinical application of this procedure is the shortage of donor islets. Clearly, a new source of functional β-cells is needed.

### SUMMARY

The present system uses optogenetics to control insulin secretion from SC-β-cells grafted in or onto an organ such as liver or kidney that would allow for automatic insulin release without manual monitoring of glucose levels. Optogenetics is a method that uses light to control select cells in living tissues. The stem cells are obtained from the patient in order to reduce rejection and transplanted within the patient's body. Additionally, a microchip will produce light to stimulate the cells to produce insulin upon detection of high glucose levels. In the following example, optogenetics is used to control of insulin secretion from stem cell derived beta cells. More specifically, an implanted microchip system is used to control Channelrhodopsin-2 (ChR2) and/or ReaChR (red-shifted ChR) transduced cells in vivo. In one aspect of the disclosure, a method for of treating type 1 diabetes includes implanting genetically modified islet cells under a capsule of or within an organ, implanting a microsystem adjacent the islet cells, said microsystem, comprising a light emitting diode stimulator comprising a plurality of light emitting diodes, determining a glucose level in a body and controlling the microsystem to selectively illuminate the islet cells to secrete insulin or glucagon or both based on the glucose level.

In another aspect of the disclosure, a system for treating type 1 diabetes in a living body comprising genetically modified islet cells includes an implantable microsystem disposed within the living body adjacent the genetically modified islet cells comprising a first portion comprising a plurality of light emitting diodes and a glucose sensor, said glucose sensor generating a glucose level signal corresponding to a glucose level within the living body. The implantable system further comprises a second portion comprising control electronics coupled to the glucose sensor and the plurality of light emitting diodes, said control electronics comprising a controller for controlling the plurality of light emitting diodes in response to the glucose level signal to increase insulin production or glucagon production at the genetically modified islet cells.

Further areas of applicability of the present disclosure will become apparent from the detailed description, the claims, and the drawings. The detailed description and specific examples are intended for purposes of illustration only and are not intended to limit the scope of the disclosure.

### DRAWINGS

The present disclosure will become more fully understood from the detailed description and the accompanying drawings.

FIG. 1 is a side view schematic representation of a small mammal having an optogenetic microsystem implanted therein.

FIG. 2A is a schematic representation of the microsystem implant of FIG. 1 in the folded position.

FIG. 2B is a side view of the microsystem implant in the unfolded position.

FIG. 2C is a side view of the microsystem in a folded position.

FIG. 2D is a high-level block diagrammatic view of the energizing module.

FIG. 2E is a detailed schematic view of the microsystem that is adjacent the energizing module through the skin.

FIG. 2F is a perspective view of an energizing system having a small mammal therein.

FIG. 2G is a front view of a human having an energization system coupled adjacent to an internal microsystem.

FIG. 3 is a flowchart of a method for operating the optogenetic microsystem.

In the drawings, reference numbers may be reused to identify similar and/or identical elements.

### DETAILED DESCRIPTION

Optogenetics is a method that uses light to control selected cells in living tissue (e.g., neurons and cardiomyocytes) that have been genetically modified to express light-sensitive ion channels and pumps. It does so by using light-sensitive proteins (e.g., ChR2) as ‘switches’ to control cellular activity. ChR2, a cation-selective channel protein permits entry of Ca²⁺ ions in response to blue light (470 nm). Optogenetics tools may be used to specifically control mouse and human β-cell functions. Insulin release from pancreatic β-cells is normally triggered by glucose, which results in membrane depolarization and opening of voltage-dependent Ca²⁺-channels followed by Ca²⁺-dependent insulin release. ChR2 opens in response to blue light, which leads to influx of extracellular Ca²⁺. Earlier studies showed that both insulinoma MIN6 (ChR2-MIN6) cells and adult mouse β-cells transduced with ChR2 secreted insulin in response to irradiation with a 470 nm laser, which was accompanied by elevated levels of intracellular Ca²⁺ (20, 21). In STZ-induced diabetic mice that were transplanted with ChR2-MIN6 cells, blue light irradiation caused significant decreases in blood glucose, and the irradiated implanted cells expressed insulin. As set forth below optogenetics is used for controlling insulin secretion in SC-β-cells differentiated from human induced pluripotent stem cells (iPSCs). Differentiation of iPSCs to p-cells in vitro, generation of ChR2 expressing human iPSCs, and differentiation of human iPSCs into islet organoids are underlying elements. Continuous blue light stimulation can significantly enhance insulin secretions from ChR2 transduced islet organoids in vitro.

Referring now to FIG. 1, a mouse is illustrated as one example of a mammal 10. Of course, humans and other mammals may benefit from the teachings set forth herein. The mouse 10 has an organ 12 such as a kidney or liver that has been exaggerated in FIG. 1. The organ 12 is enclosed by a capsule 14. The capsule 14 has islet cells 16 implanted thereon. The islet cells 16 may also be referred to an islet organoid that derived from human iPSCs. For other mammals such as a human the organ 12 may be a liver with the islet cells transplanted therein through a portal vein. The islet cells 16 and the generation of the islet organoids are described in further detail below. The islet cells 16 are used to produce insulin for used in the body of the mammal 10, which also has a peritoneum 18.

Insulin is generated in response to optogenetic stimulation from a microsystem implant 20 (implantable microsystem). The microsystem implant 20 may take various forms. In one example, the microsystem implant 20 is a wireless opto-electro-chemo microsystem (WOECM) that is implanted within the mammal 10. In the present example, the microsystem implant 20 is secured to the peritoneum 18. Ultimately, the microsystem implant 20 is adjacent to the islet cells 16 to optogenetically stimulate the cells 16.

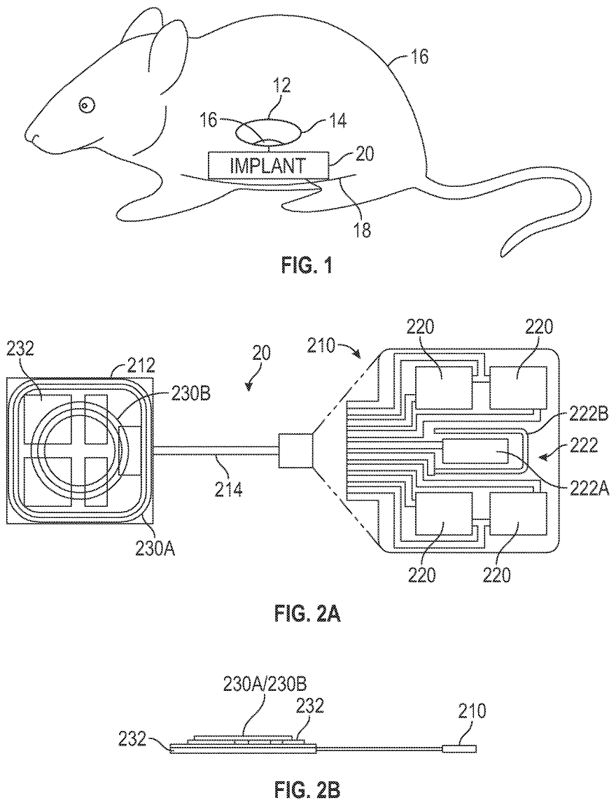

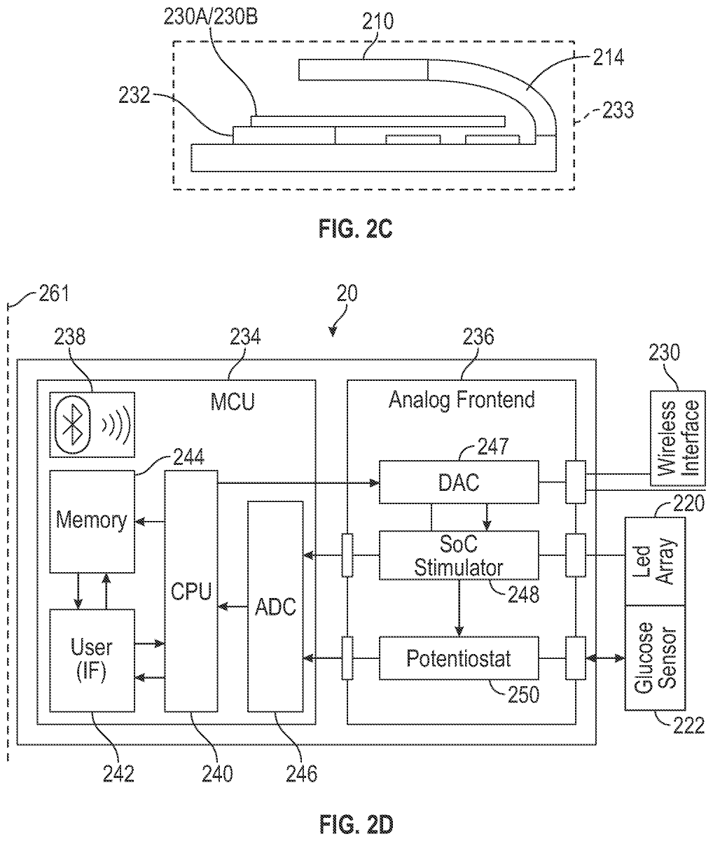

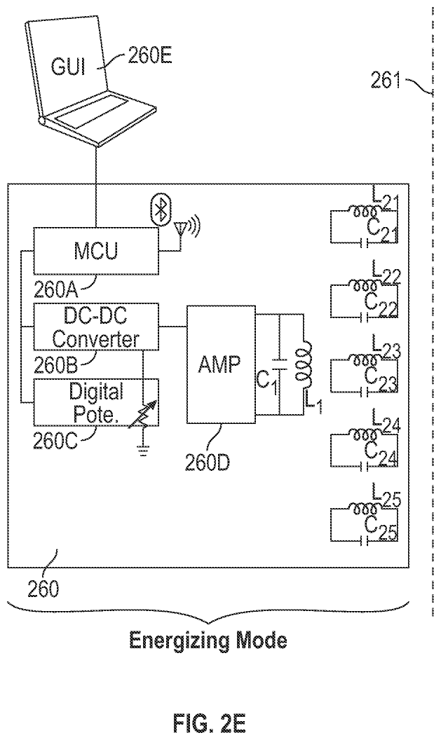

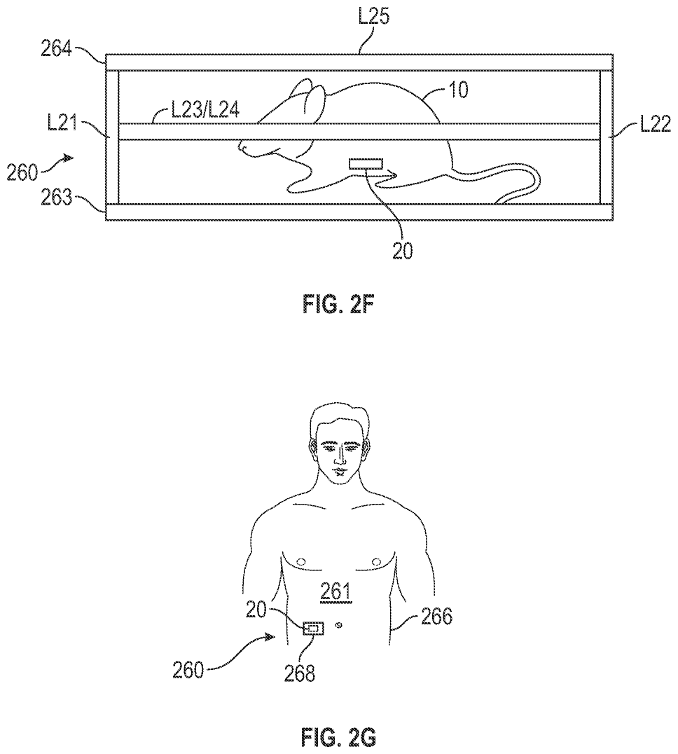

Referring now to FIGS. 2A-2F, the microsystem implant 20 of FIG. 1 is forth in further detail in which in-situ glucose sensing and closed-loop optical stimulation of islet cells is performed. That is, an implanted microchip system is used to control ChR2 transduced cells in vivo.

A patch 210 is coupled to a circuit substrate 212 by an interconnection such as a flexible cable 214. That is, the patch 210 is separated from the circuit substrate 212 by the flexible cable 214 The patch 210 in this example comprises four individually addressable microscale light emitting diode (μLEDs) 220 and a glucose sensor 222. The patch 210 in this example comprises a 1.5×1.5 mm² flexible polyimide substrate. Each μLED 220 in this example provides an emission area of 220×250 μm² using an array of LEDs to maximize the volumetric coverage of optical illumination. The LEDs 220 may be integrated with two different wavelengths to provide different colors blue LED (470 nm) in a first LED in the plurality of LEDs for activation of ChR2 and red LEDs (640 nm) in a second LED of the plurality of LEDs for activation of ReaChR (red-shifted ChR), which control cell types transfected with different light-gated ion channels for hormones secretion. The μLEDs 220 may be operated in differently for different mammals. Continuous operation of the LEDs, or selective operation may be performed. The LEDs may be pulse width modulated over a time period until the glucose sensor determines a reduction in glucose due to the production of insulin from the stimulated implanted islet cells.

A working electrode (WE) 222A has enzymes disposed thereon. The working electrode 222A is disposed adjacent to a counter electrode 222B. A widely used enzyme associated with glucose metabolism is glucose oxidase (GOx) that yields gluconic acid and hydrogen peroxide. Of course, the size of the sensor, the composition and amount of enzymes may change depending on the mammal. The material and structural design of the working electrode 222A may be configured to simultaneously achieve high selectivity, sensitivity, and stability of the glucose sensor. Electrodes treated with nanoparticles (i.e., platinum nanoparticles) or nanostructures can effectively enhance the selectivity and sensitivity of GOx based sensors. Electro-polymerization to entrap GOx while excluding redox interferences and protein macromolecules may also be used.

A scalable, wafer-level method for sensor fabrication, involving multiple steps of polymerization, photolithography, metallization, and lift-off may be used. Gold may be used as the electrode material because of its relatively high conductivity, biocompatibility, and chemical resistance. Polyimide or Parylene C suitable substrate materials, which may be patterned using oxygen plasma etching. The sensitivity and linearity of the GOx-based sensor with different coating materials and nanostructures in NaCl solutions spiked with glucose at various concentrations may be calibrated. To test the specificity of the sensor, measurements using samples spiked with glucose and other common metabolites, such as uric acid, lactate and creatinine.

The circuit substrate 212 has a wireless interface that includes a transmitting coil 230A and a receiving coil 230B. Control electronics 232 are also coupled to the circuit substrate 212. Although a wireless type of system is set forth, the system may also have a battery to power the microsystem implant 20. Further, the microsystem implant 20 may also be encapsulated. That is, the implant 20 may be completely encapsulated and energized through the receiving coil 230B when a sufficient electrical field is present. Of course, at least the glucose sensor may be exposed to measure glucose in the bodily fluid.

Referring now to FIG. 2D, in general, the system has a microcontroller 234 that is in communication with an analog front end 236. The analog front end 236 is in communication with the wireless interface 230 that includes the transmit coil, 230A and the receiving coil 230B. The analog front end 236 is also in communication with the LED array 220 and the glucose sensor 222.

A Bluetooth® transceiver 238 is included within the microcontroller 234. The Bluetooth® transceiver 238 is a communication controller. Of course, other techniques for communicating signals to and from the microcontroller 234 may be used. Bluetooth® was chosen in this example for its convenience.

The microcontroller 234 also includes a central processing (CPU) that is microprocessor-based and is programmed to perform the various steps and functions described in this disclosure. The CPU 240 is in communication with a user interface 242 and a memory 244. The user interface 242 allows a user external to the mammal to control various functions therein. An analog to digital converter 246 is also disposed within the microcontroller 234. The analog to digital converter 246 receives data from a system on-ship (SoC) stimulator 248 disposed within the analog front end 236. The stimulator 248 is in communication with the LED array 220 and operates the LED array 220. The LED array 220, in this example, generates blue light at a wavelength of 470 nm using, in this example, four LED arrays.

A digital to analog converter (DAC) couples the CPU 240 to provide power to the wireless interface 230.

The row and column of the LEDs within the LED array is controlled by the stimulator 248 which is ultimately controlled by the CPU 240. A potentiostat 250 is used to control the data to and from the glucose sensor 222. One example of a potentiostat chip is a Texas Instruments LMP 9100.

The potentiostat 250 performs low-power chronoamperometric measurements of the GOx-based sensor in 2-, 3-, or 4-electrode cell configurations, generating an output voltage proportional to the cell current in response to various glucose concentrations. The MCU 234 configures the parameter setup of the potentiostat 250. The amplified glucose sensing signals are be sampled and digitized by the built-in analog-to-digital converter (ADC) 246 of the MCU 234. The digitized glucose level signals are passed to the central processing unit 240 (CPU) of the MCU, where the signal processing algorithms of the closed-loop control of optogenetic stimulation are running. The MCU 234 extracts the sensor current output and correlate it to the glucose levels based on pre-set calibration curves. Using the measured glucose profile, the MCU 234 generates and send stimulation commands to the optical stimulator 248 to efficiently control the μLED arrays 220 for optogenetics stimulation. The stimulator 248 employs the switched-capacitor-based stimulation (SCS) architecture, which is a much safer and yet more efficient method for applying optical stimulation.

Referring now also to FIGS. 2E-2G an energy transmission cage is used in this example as an energizing system 260 that drives the opto-electro-chemo patch microsystem implant 20 through the skin 261 of a mammal. The energizing system 260 fits a small mammal therein. For a human or other mammals, the energizing system 260 may be an external device such as patch, belt or device worn externally adjacent to the implanted microsystem to allow energization thereof through the skin 261 of the mammal.

The microsystem implant 20 integrates an analog front-end (AFE) potentiostat 250 (i.e., Texas Instruments, LMP91000) with a wireless optogenetics system-on-chip (SoC) stimulator 248. The microsystem implant 20 comprises, the Bluetooth® transceiver 238 (e.g., nRF52 MCU), on a flexible polyimide circuit board or circuit substrate 212. The potentiostat 250 allows low-power chronoamperometric measurements of the GOx-based sensor in 2-, 3-, or 4-electrode cell configurations, generating an output voltage proportional to the cell current signal in response to different glucose concentrations. The MCU 234 configures the parameter setup of the potentiostat 250 through an I2C interface. The amplified glucose sensing signals are sampled and digitized by the built-in analog-to-digital converter (ADC) 246 of the MCU 234. The digitized signal is passed to the central processing unit (CPU) 240 of the MCU 238, where the signal processing algorithms of the closed-loop control of optogenetic stimulation is running. The MCU 234 extracts the sensor current output and correlate it to the glucose levels based on pre-set calibration curves. The Bluetooth® transceiver (transmitter/receiver) 238 communicates externally to and from the microsystem implant 20. Of course, other types of transceiver technology may be used. Using the measured glucose profile, the MCU 234 generates and sends stimulation commands to stimulator SoC 248 to efficiently control the μLED arrays 220 for optogenetics stimulation. The SoC 248 employs the switched-capacitor based stimulation (SCS) architecture, which is a much safer and yet more efficient method for applying optical stimulation. The timing control blocks generates reference clocks and stimulating pulses, which is adjustable in pulse width and duty cycle.

A sufficient amount of wireless power is used to power the μLEDs through near-field electromagnetic coupling, which inherently requires high instantaneous power, thus increasing the electromagnetic field intensity to very high and potentially unsafe levels. To address this challenge, the PTE of every stage of the power delivery path from the power source to the, enables low-temperature operation well below the specific absorption rate (SAR) limit.

Referring now also to FIG. 2E and 2F, in the present example the energizing system for a small mammal is used. The mammal is housed in the energizing system 260. For larger mammals such as a human, a wearable device may incorporate the energizing module 260 therein. The energizing system 260 has a transmitter coil L₁, and the power control electronics housed in a driver box 263 underneath a cage 264 and has transmitter resonators L₂₁-L₂₅ wrapping around the cage. An MCU 260A is in the driver box 263. Likewise, a DC-to-DC converter 260B, a digital potentiometer 260C and a power amplifier 260D are used to power and control the energizing system 260 ultimately to wirelessly power the microsystem implant 20. The graphical user interface (GUI) 260E provides a way to control the system and obtain data externally. The GUI 260E may be a computer, touch screen or another type of controlling device. The closed-loop power control (CLPC) loop implemented in the energizing system 260 adjusts the transmitted power to stabilize the delivered power at a level that is just enough to reliably operate the microsystem implant 20 despite coil misalignments and distance variations. Referring now to FIG. 2G, a human 266 (or other mammals) may have the energizing system 260 as an external device 268 such as patch, belt, electrically conductive tattoo or device worn externally adjacent to the microsystem implant 20 to allow energization thereof through the skin 261 of the mammal.

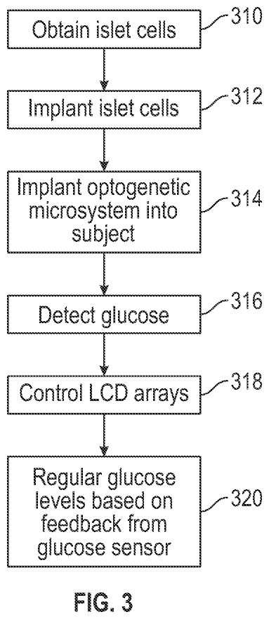

Referring now to FIG. 3, a method for operating the system is set forth. Islet cells for transplanting are obtained for transplant in step 310. Ulimately the islet cells 312 are implanted into the mammal. In this example, the islet cell implantation occurs at the kidney and more specifically under the capsule 14 of the kidney or within the liver. The cells for islet transplantation can be derived from the subject or from a donor. Optogenetics is used to control of insulin secretion from stem cell derived beta cells. The transplanted grafts survive over the years. Stem cell differentiation technology has also greatly advanced in recent years, resulting in clinical trials using stem cell-based therapies for patients with diabetes. In vitro differentiation protocols that convert pluripotent stem cells into pancreatic β-cells have been developed. Studies that have recently advanced to Phase I trials successfully demonstrate the application of human embryonic stem cell (hESC)-derived pancreatic progenitors for restoring normoglycemia of T1 D patients. While promising, a challenge in producing any cell type in vitro is the heterogeneity of the cells generated by directed differentiation. At each step of the process, some cells follow the desired path, while others stray. Some cells end up producing both insulin and/or glucagon, and the identity of these poly-hormonal cells is controversial. Insulin release is also influenced by other cell types in pancreatic islets including α- and delta-cells through a number of paracrine mechanisms. Although SC-β-cells are highly similar to endogenous β-cells, the two key problems preventing the effective use of SC-β-cells in clinical therapies are associated with the lack of glucose responsiveness in SC-β-cells and low efficiency of insulin secretion. SC-β-cells do not express transcription factors UCN3, MAFA and SIX3, meaning that these insulin producing cells may not respond to high glucose levels properly. Compared to endogenous islets, purified SC-β-cells show lower levels of insulin secretion in glucose-stimulated insulin secretion (GSIS) assays in vitro.

To synchronize insulin secretion of differentiated cells with the rising glucose levels and improve efficacy, a closed-loop controlled, wireless opto-electro-chemo microsystem (WOECM) is implanted into the subject. In the present example the microsystem implant twenty was implanted on the peritoneum adjacent to the kidney and more specifically, the grafted islet cells/islet organoids under the kidney capsule. In the case of a liver the implant twenty is implanted on the peritoneum adjacent to the liver and more specifically adjacent to the islet cells/islet organoids implanted into the liver through a portal vein. The microsystem implant twenty consists of enzymatic glucose sensors to continuously and accurately measure the spontaneous changes in glucose concentrations under the peritoneum.

In step 316, the amount of insulin adjacent to the glucose sensor is determined using the glucose sensor using closed-loop feedback to the microcontroller.

Using the monitored glucose profile, step 318 uses a closed-loop LED stimulator 248 that is controlled to control insulin release from SC-β-cells expressing ChR2, to regulate in situ glucose levels in step 320. To achieve these goals, ChR2 is stably expressed under the insulin promoter in human iPSCs. The ChR2-transduced iPSCs are differentiated into β-cells in vitro using a standardized protocol (SA-1). For mimicking pulsatile insulin release, islet cells are irradiated using pulses of light, for example, every thirties for half an hour. The amount of light may be tuned and verified for different mammals relative to the output intensity and stability of the LED illumination using an optical power meter as necessary to optimize the efficiency of optogenetic stimulation. LED wavelength is switchable from blue (470 nm) to red light (640 nm) for activation of ReaChR that is stable expressed under the glucagon promoter in human iPSCs differentiated α-cells to prevent hypoglycemia as well. That is, the microsystem and the LEDs therein may be wavelength controlled to selectively illuminate the islet cells to secrete or produce insulin or glucagon or both based on the glucose level

The foregoing description of the embodiments has been provided for purposes of illustration and description. It is not intended to be exhaustive or to limit the disclosure. Individual elements or features of an embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

The term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip. While various embodiments have been disclosed, other variations may be employed. All of the components and function may be interchanged in various combinations. It is intended by the following claims to cover these and any other departures from the disclosed embodiments which fall within the true spirit of this invention.

What is claimed is:

1. A method of treating type 1 diabetes comprising;
implanting genetically modified islet cells under a capsule of or within an organ;
implanting a microsystem adjacent the islet cells, said microsystem, comprising a light emitting diode stimulator comprising a plurality of light emitting diodes;
determining a glucose level in a body; and
controlling the microsystem to selectively illuminate the islet cells to secrete insulin or glucagon or both based on the glucose level.

2. The method of claim 1 wherein implanting islet cells comprises implanting islet cells under a capsule of an organ.

3. The method of claim 1 wherein implanting islet cells comprises implanting islet cells within an organ.

4. The method of claim 1 wherein implanting the microsystem adjacent the islet cells comprise implanting the microsystem adjacent the islet cells on a peritoneum.

5. The method of claim 1 wherein controlling the microsystem to selectively illuminate islet cells comprises controlling a first light emitting diode of the plurality of light emitting diodes to generate a first wavelength different than a second wavelength of a second light emitting diode of the plurality of light emitting diodes.

6. The method of claim 5 wherein controlling comprises controlling the first light emitting diode to generate blue light and the second light emitting diode to generate red light.

7. The method of claim 6 further comprising activating ChR2 with the first light emitting diode and activating ReaChR with the second light emitting diode.

8. The method of claim 1 determining a glucose level in a body from a glucose sensor disposed within the microsystem.

9. The method of claim 1 determining a glucose level in a body from a glucose sensor disposed adjacent to the light emitting diodes of the microsystem.

10. A system for treating type 1 diabetes in a living body comprising genetically modified islet cells comprising:
an implantable microsystem disposed within the living body adjacent the genetically modified islet cells comprising a first portion comprising a plurality of light emitting diodes and a glucose sensor, said glucose sensor generating a glucose level signal corresponding to a glucose level within the living body; and
said implantable microsystem comprising a second portion comprising control electronics coupled to the glucose sensor and the plurality of light emitting diodes, said control electronics comprising a controller for controlling the plurality of light emitting diodes in response to the glucose level signal to increase insulin production or glucagon production at the genetically modified islet cells.

11. The system of claim 10 wherein the islet cells are implanted under a capsule or within an organ.

12. The system of claim 10 wherein the implantable microsystem is coupled to a peritoneum of the living body.

13. The system of claim 10 wherein the glucose sensor is coupled to a potentiostat for generating an analog signal corresponding to a glucose level.

14. The system of claim 13 further comprising an analog to digital converter communicating a digitized glucose level signal to the controller based on the analog signal.

15. The system of claim 10 wherein a first light emitting diode of the plurality of light emitting diodes generates a first wavelength different than a second wavelength of a second light emitting diode of the plurality of light emitting diodes.

16. The system of claim 15 wherein the first light emitting diode generates blue light for activating ChR2 and the second light emitting diode generates red light for activating ReaChR.

17. The system of claim 10 wherein the first portion and the second portion are separated by a flexible cable.

18. The system of claim 10 further comprising a receiving coil disposed within the implantable microsystem for energizing the implantable microsystem from energy received from an energizing module.

19. The system of claim 18 further comprising a transmitting coil coupled to the controller for transmitting data to an energizing module.

20. The system of claim 19 wherein the transmitting coil is coupled to a Bluetooth® transceiver.
